(19) United States
(12) Patent Application Publication
(10) Pub. No.: US 2011/0282368 A1
(43) Pub. Date: Nov. 17, 2011

(54) Fistula Repair Device with Extendable Barbs and Therapeutic Cell Delivery

(76) Inventors: Jeffrey S. Swayze, Hamilton, OH (US); Foster B. Stulen, Mason, OH (US); Mark H. Ransick, West Chester, OH (US); Steven G. Hall, Cincinnati, OH (US); John A. Hibner, Mason, OH (US); Julia J. Hwang, Wayland, MA (US); Yolanda F. Carter, Union, KY (US); Frederick E. Shelton, IV, Hillsboro, OH (US)

(21) Appl. No.: 12/778,587

(22) Filed: May 12, 2010

(51) Int. Cl. A61B 17/22; A61B 17/00

(52) U.S. Cl. 606/159; 606/213

(57) ABSTRACT

A fistula repair system includes a debriding member that may be inserted into a fistula to debride the fistula wall. The debriding member may include a plurality of selectively extendable barbs. The barbs may be hollow to deliver a medical fluid within the fistula. The debriding member may also include a plurality of openings configured to communicate the medical fluid within the fistula. The barbs may extend through the openings. The openings may include associated protrusions that are configured to debride the fistula wall. The medical fluid may include a slurry of tissue and a scaffold material. The tissue in the slurry may comprise minced tissue that was harvested from the patient afflicted with the fistula. A balloon may be used to at least temporarily seal one end of the fistula. An RF probe or mechanical hook member may be used to seal the other end of the fistula.

### BACKGROUND

Fistulae can occur for a variety of reasons, such as, from a congenital defect, as a result of inflammatory bowel disease such as Crohn's disease, some sort of trauma, or as a side effect from a surgical procedure. Additionally, several different types of fistulae can occur in humans, for example, urethro-vaginal fistulae, vesico-vaginal fistulae, tracheo-esophageal fistulae, gastrointestinal fistulae, for example gastrocutaneous, enterocutaneous and colocutaneous fistulae, and any number of anorectal fistulae such as recto-vaginal fistula, recto-vesical fistulae, recto-urethral fistulae, and recto-prostatic fistulae. When fistulas form, they can track between intestinal segments or between an intestinal segment and other organs (e.g., bladder, vagina, etc.), adjacent tissue, or the skin. Fistulas are classified as internal when they communicate with adjacent organs (e.g., entero-enteric and rectovaginal fistulas, etc.) and external when they communicate with the dermal surface (e.g., enterocutaneous, peristomal and perianal fistulas, etc.).

Promoting and improving tissue healing around the fistula opening and in the fistula tract may be an important aspect of fistulae medical treatments. For instance, promoting and improving tissue healing may lead to quicker recovery times and lessen the opportunity for infection, particularly in a post-surgical context. Some advancements in the medical arts pertaining to systems, methods, and devices to promote and improve tissue healing in patients aim to add active biological components (e.g., tissue particles, stem cells, other types of cells, etc.) to a wound site (e.g., surgical site, accidental trauma site, etc.) or other defect site (e.g., caused by disease or other condition, etc.) to promote tissue regeneration or accelerate tissue healing. When adding biological components to a site, such components may be added independently or as part of a specifically designed matrix or other mixture depending on the condition being treated and goals of the treatment. Some examples of cell-based therapy technology are disclosed in U.S. Pub. No. 2008/0311219, entitled “Tissue Fragment Compositions for the Treatment of Incontinence,” published Dec. 18, 2008, the disclosure of which is incorporated by reference herein. Additional examples of cell-based therapy technology are disclosed in U.S. Pub. No. 2004/0078090, entitled “Biocompatible Scaffolds with Tissue Fragments,” published Apr. 22, 2004, the disclosure of which is incorporated by reference herein. Additional examples of cell-based therapy technology are disclosed in U.S. Pub. No. 2008/0071385, entitled “Conformable Tissue Repair Implant Capable of Injection Delivery,” published Mar. 20, 2008, the disclosure of which is incorporated by reference herein.

Regardless of how the active biological components are delivered or applied to a site, the biological components must first be obtained and prepared. One approach for obtaining such biological components is to harvest the desired components from a healthy tissue specimen (e.g., in an adult human). Examples of devices and associated methods for collecting and processing harvested tissue are disclosed in U.S. Pub. No. 2004/0193071, entitled “Tissue Collection Device and Methods,” published Sep. 30, 2004, the disclosure of which is incorporated by reference herein. Additional examples of devices and associated methods for collecting and processing harvested tissue are disclosed in U.S. Pub. No. 2005/0038520, entitled “Method and Apparatus for Resurfacing an Articular Surface,” published Feb. 17, 2005, the disclosure of which is incorporated by reference herein. Additional examples of devices and associated methods for collecting and processing harvested tissue are disclosed in U.S. Pat. No. 7,611,473, entitled “Tissue Extraction and Maceration Device,” issued Nov. 3, 2009, the disclosure of which is incorporated by reference herein. Additional examples of devices and associated methods for collecting and processing harvested tissue are disclosed in U.S. Pub. No. 2008/0234715, entitled “Tissue Extraction and Collection Device,” published Sep. 25, 2008, the disclosure of which is incorporated by reference herein. Additional examples of devices and associated methods for processing harvested tissue are disclosed in U.S. Pub. No. 2005/0125077, entitled “Viable Tissue Repair Implants and Methods of Use,” published Jun. 9, 2005, the disclosure of which is incorporated by reference herein. Additional examples of devices and associated methods for collecting and processing harvested tissue are disclosed in U.S. Pat. No. 5,694,951, entitled “Method for Tissue Removal and Transplantation,” issued Dec. 9, 1997, the disclosure of which is incorporated by reference herein. Additional examples of devices and associated methods for collecting and processing harvested tissue are disclosed in U.S. Pat. No. 6,990,982, entitled “Method for Harvesting and Processing Cells from Tissue Fragments,” issued Jan. 31, 2006, the disclosure of which is incorporated by reference herein. Additional examples of devices and associated methods for collecting and processing harvested tissue are disclosed in U.S. Pat. No. 7,115,100, entitled “Tissue Biopsy and Processing Device,” issued Oct. 3, 2006, the disclosure of which is incorporated by reference herein.

Once harvested and suitably processed (e.g., incorporated with a scaffold, etc.), biological material such as tissue fragments may be applied to a wound site or other type of site within the human body in a variety of ways. Various methods and devices for applying such biological material are disclosed in one or more of the U.S. patent references cited above. Additional methods and devices for applying such biological material are disclosed in U.S. Pub. No. 2005/0113736, entitled “Arthroscopic Tissue Scaffold Delivery Device,” published May 26, 2005, the disclosure of which is incorporated by reference herein.

While a variety of devices and techniques may exist for harvesting, processing, and applying biological components from a tissue specimen, it is believed that no one prior to the inventor(s) has made or used an invention as described herein.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings. In the drawings, like numerals represent like elements throughout the several views.

FIG. 1 depicts a perspective view of the distal end of an exemplary version of a fistula repair device;

FIG. 2 depicts another perspective view of the fistula repair device of FIG. 1, with barbs extended;

FIG. 3 depicts another perspective view of the fistula repair device of FIG. 1, with barbs extended and releasing a therapeutic material;

FIG. 4 depicts another perspective view of the fistula repair device of FIG. 1, releasing a therapeutic material and with barbs retracted;

FIG. 5 depicts perspective view of an exemplary alternative version of a fistula repair device, with a stent;

FIG. 6 depicts another perspective view of the fistula repair device of FIG. 5, with a stent and with barbs extended;

FIG. 7 depicts a front interior view of an exemplary version of a fistula repair device being inserted into a rectum;

FIG. 8 depicts a perspective view of the fistula repair device of FIG. 7 entering a fistula;

FIG. 9 depicts a perspective view of the fistula repair device of FIG. 7 engaging the fistula with extended barbs;

FIG. 10 depicts a perspective view of the fistula repair device of FIG. 7 engaging the fistula with the barbs extending farther into the wall of the fistula;

FIG. 11 depicts a cross sectional view of the fistula repair device of FIG. 7 engaging the fistula with the extended barbs and releasing a therapeutic material in the fistula;

FIG. 12 depicts a cross sectional view of a stent from the fistula repair device of FIG. 7 positioned in the fistula;

FIG. 13 depicts a perspective view of an exemplary alternative version of a fistula repair device;

FIG. 14 depicts a perspective view of an exemplary version of a plug formed in a fistula by a fistula repair device;

FIG. 15 depicts a front interior view of an exemplary version of a fistula repair device being inserted into a fistula;

FIG. 16 depicts a front, partially interior view of the fistula repair device of FIG. 15 inserted into the fistula;

FIG. 17 depicts a front, partially interior view of the fistula repair device of FIG. 15 inserted into the fistula and inflating a balloon;

FIG. 18 depicts a perspective view of the fistula repair device of FIG. 15 inserted into the fistula with the balloon inflated;

FIG. 19 depicts a perspective view of an exemplary alternative version of a fistula repair device, with a hook;

FIG. 20 depicts a perspective view of an exemplary alternative version of a fistula repair device, with a radio frequency sealer;

FIG. 21 depicts an end view of an exemplary version of a fistula repair device;

FIG. 22 depicts a side perspective view of the fistula repair device of FIG. 21;

FIG. 23 depicts a front, interior view of the fistula repair device of FIG. 21 inserted into a rectum and approaching a fistula;

FIG. 24 depicts a perspective view of the fistula repair device of FIG. 21 with an outer sheath inserted into the fistula;

FIG. 25 depicts a perspective view of the fistula repair device of FIG. 21 with the outer sheath inserted into the fistula and a therapeutic material being released from the outer sheath; and

FIG. 26 depicts a perspective view of the fistula repair device of FIG. 21 with an RF sealer sealing the fistula.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples should not be used to limit the scope of the present invention. Other features, aspects, and advantages of the versions disclosed herein will become apparent to those skilled in the art from the following description, which is by way of illustration, one of the best modes contemplated for carrying out the invention. As will be realized, the versions described herein are capable of other different and obvious aspects, all without departing from the invention. Accordingly, the drawings and descriptions should be regarded as illustrative in nature and not restrictive.

I. Overview of Exemplary Treatment Compositions, Devices, and Methods

Examples described herein include devices that are operable to harvest tissue, mince or morcellate tissue, mix tissue particles with other medical fluid components, and/or dispense a medical fluid at a target site in a patient. As described in greater detail below, the medical fluid may include any of a variety of biocompatible materials that accelerate tissue healing, promote tissue regeneration, and/or provide other results. As used herein, the terms “tissue treatment composition,” “tissue repair composition,” and “medical fluid” should be read interchangeably. It should also be understood that a tissue treatment composition or medical fluid as referred to herein may have any suitable consistency, including but not limited to the consistency of a slurry.

A medical fluid as referred to herein may be derived from any biocompatible material, including but not limited to synthetic or natural polymers. The consistency of the medical fluid may be viscous, or gel-like, that of a slurry composed of microparticles, or any other suitable consistency. By way of example only, any fluid consistency that may permit injection through a catheter may be used. The medical fluid may also provide adhesive characteristics, such that once it is injected at a target site (e.g., into a fistula), the fluid coagulates or gels (e.g., allowing for a plug to be retained within a fistula). The medical fluid of the present example is also able to support cell migration and proliferation such that healing at a target site in a patient can occur. The fluid is suitable to be mixed with biological materials. Examples of medical fluid components include but are not limited to thrombin, platelet poor plasma (PPP) platelet rich plasma (PRP), starch, chitosan, alginate, fibrin, polysaccharide, cellulose, collagen, gelatin-resorcin-formalin adhesive, oxidized cellulose, mussel-based adhesive, poly (amino acid), agarose, amylose, hyaluronan, polyhydroxybutyrate (PHB), hyaluronic acid, poly(vinyl pyrrolidone) (PVP), poly(vinyl alcohol) (PVA), polylactide (PLA), polyglycolide (PGA), polycaprolactone (PCL), and their copolymers, VICRYL® (Ethicon, Inc., Somerville, N.J.), MONOCRYL material, PANACRYL (Ethicon, Inc., Somerville, N.J.), and/or any other material suitable to be mixed with biological material and introduced to a wound or defect site, including combinations of materials. Other suitable compounds, materials, substances, etc., that may be used in a medical fluid will be apparent to those of ordinary skill in the art in view of the teachings herein.

By way of example only, one or more components in a medical fluid or tissue treatment composition may comprise at least one viable tissue fragment having one or more viable cells that, once applied, can proliferate and integrate with tissue at a target site in a patient. For instance, viable cells may migrate out of a tissue particle and populate a scaffold material, which may be positioned at a target site in a patient. Such tissue fragments may have been harvested from the same patient in whom they are reapplied; or may have been harvested from another person or source. The tissue fragments may comprise autogenic tissue, allogenic tissue, xenogenic tissue, mixtures of any of the foregoing, and/or any other type(s) of tissue. The tissue fragments may include, for example, one or more of the following tissues or tissue components: stem cells, cartilage tissue, meniscal tissue, ligament tissue, tendon tissue, skin tissue, muscle tissue (e.g., from the patient's thigh, etc.), periosteal tissue, pericardial tissue, synovial tissue, fat tissue, bone marrow, bladder tissue, umbilical tissue, embryonic tissue, vascular tissue, blood and combinations thereof. Of course, any other suitable type of tissue may be used, including any suitable combination of tissue types. In some versions, the type of tissue used is selected from a tissue type most resembling the tissue at, near, or surrounding the target site (e.g., fistula, etc.).

Tissue for providing at least one viable tissue fragment may be obtained using any of a variety of tissue biopsy devices or using other types of tissue harvesting devices or techniques. Exemplary biopsy devices include those taught in U.S. Pat. No. 5,526,822, entitled “Method and Apparatus for Automated Biopsy and Collection of Soft Tissue,” issued Jun. 18, 1996; U.S. Pat. No. 6,086,544, entitled “Control Apparatus for an Automated Surgical Biopsy Device,” issued Jul. 11, 2000; U.S. Pub. No. 2007/0118048, entitled “Remote Thumbwheel for a Surgical Biopsy Device,” published May 24, 2007; U.S. Pub. No. 2008/0214955, entitled “Presentation of Biopsy Sample by Biopsy Device,” published Sep. 4, 2008; U.S. Non-Provisional patent application Ser. No. 12/337,942, entitled “Biopsy Device with Central Thumbwheel,” filed Dec. 18, 2008; and U.S. Non-Provisional patent application Ser. No. 12/483,305, entitled “Tetherless Biopsy Device with Reusable Portion,” filed Jun. 12, 2009. The disclosure of each of the above-cited U.S. patents, U.S. patent application Publications, and U.S. Non-Provisional patent applications is incorporated by reference herein. Such biopsy devices may be used to extract a plurality of tissue specimens from one or more sites in a single patient. It should also be understood that any suitable device described in any other reference that is cited herein may be used to harvest tissue. Additional examples of devices that may be used to harvest tissue will be described in greater detail below. Other examples will be apparent to those of ordinary skill in the art in view of the teachings herein. Tissue harvesting sites may include the same sites in which tissue is reapplied as part of a treatment. In addition or in the alternative, tissue may be harvested from one site and then reapplied at some other site as part of a treatment. In some versions, the tissue is reapplied in the same patient from whom the tissue was originally harvested. In some other versions, the tissue is applied in a patient who is different from the patient from whom the tissue was originally harvested.

A tissue specimen may be obtained under aseptic conditions, and then processed under sterile conditions to create a suspension having at least one minced, or finely divided, tissue fragment. In other words, harvested tissue may be diced, minced or morcellated, and/or otherwise processed. Harvested tissue specimens may be minced and otherwise processed in any of a variety of ways. For instance, examples of tissue mincing and processing are described in U.S. Pub. No. 2004/0078090, the disclosure of which is incorporated by reference herein. Alternatively, merely exemplary non-conventional devices and techniques that may be used to mince and process tissue will be described in greater detail below, while other examples will be apparent to those of ordinary skill in the art in view of the teachings herein. In order to ensure viability of the tissue, agitators or other features of a mincing and/or mixing device may be designed to sever and mix (rather than crush or compress) the tissue. In some settings, tissue specimens may be minced and/or mixed in a standard cell culture medium, either in the presence or absence of serum. Tissue fragments may also be contacted with a matrix-digesting enzyme to facilitate cell migration out of an extracellular matrix surrounding the cells. Suitable matrix-digesting enzymes that may be used in some settings include, but are not limited to, collagenase, chondroitinase, trypsin, elastase, hyaluronidase, peptidase, thermolysin, and protease. The size of each tissue fragment may vary depending on the target location, method for delivering the treatment composition to the target site, and/or based on various other considerations. For example, the tissue fragment size may be chosen to enhance the ability of regenerative cells (e.g., fibroblasts) in the tissue fragments to migrate out of the tissue fragments, and/or to limit or prevent the destruction of cell integrity. In some settings, ideal tissue fragments are between approximately 200 microns and approximately 500 microns in size. As another merely illustrative example, ideal tissue fragments may be sized within the range of approximately 0.05 mm³ and approximately 2 mm³; or more particularly between approximately 0.05 mm³ and approximately 1 mm³. Of course, various other tissue fragment sizes may be ideal in various different settings.

In some versions, a medical fluid may comprise minced tissue fragments suspended in a biocompatible carrier. Suitable carriers may include, for example, a physiological buffer solution, a flowable gel solution, saline, and water. In the case of gel solutions, the tissue repair composition may be in a flowable gel form prior to delivery at the target site, or may form a gel and remain in place after delivery at the target site. Flowable gel solutions may comprise one or more gelling materials with or without added water, saline, or a physiological buffer solution. Suitable gelling materials include biological and synthetic materials. Exemplary gelling materials include the following: proteins such as collagen, collagen gel, elastin, thrombin, fibronectin, gelatin, fibrin, tropoelastin, polypeptides, laminin, proteoglycans, fibrin glue, fibrin clot, platelet rich plasma (PRP) clot, platelet poor plasma (PPP) clot, self-assembling peptide hydrogels, Matrigel or atelocollagen; polysaccharides such as pectin, cellulose, oxidized regenerated cellulose, chitin, chitosan, agarose, or hyaluronic acid; polynucleotides such as ribonucleic acids or deoxyribonucleic acids; other materials such as alginate, cross-linked alginate, poly(N-isopropylacrylamide), poly(oxyalkylene), copolymers of poly(ethylene oxide)-poly(propylene oxide), poly(vinyl alcohol), polyacrylate, or monostearoyl glycerol co-Succinate/polyethylene glycol (MGSA/PEG) copolymers; and combinations of any of the foregoing. In addition to providing a flowable carrier solution for tissue fragments, a gelling agent(s) may also act as an adhesive that anchors the tissue repair composition at the target site. In some versions, an additional adhesive anchoring agent may be included in the tissue repair composition or medical fluid. Also, one or more cross-linking agents may be used in conjunction with one or more gelling agents in order to cross-link the gelling agent.

The concentration of tissue fragments in a carrier and/or one or more medical fluid components may vary depending on the target site location, method for delivering the treatment composition to the target site, and/or for various other reasons. By way of example, the ratio of tissue fragments to carrier (by volume) may be in the range of about 2:1 to about 6:1, or in the range of about 2:1 to about 3:1. The medical fluid may also include one more additional healing agents, such as biological components that accelerate healing and/or tissue regeneration. Such biological components may include, for example, growth factors, proteins, peptides, antibodies, enzymes, platelets, glycoproteins, hormones, cytokines, glycosaminoglycans, nucleic acids, analgesics, viruses, isolated cells, or combinations thereof. The medical fluid may further include one or more additional treatment components that prevent infection, reduce inflammation, prevent or minimize adhesion formation, and/or suppress the immune system. In some versions where a scaffold is used in conjunction with a tissue treatment composition, one or more of these additional biological components or additional treatment components may be provided on and/or within the scaffold. Similarly, in some versions where a scaffold plug is used in conjunction with a tissue repair composition, one or more of these additional biological components or additional treatment components may be provided on and/or within the scaffold plug. Some examples described herein may also include one or more adhesive agents in conjunction with viable tissue fragments.

As noted above, the harvested tissue may be combined with a scaffold material and/or other substances as part of a medical fluid, as described herein, for administration to the patient. To the extent that tissue is incorporated with a scaffold material, it should be understood that any suitable material or combination of materials may be used to provide a scaffold. By way of example only, scaffold material may include a natural material, a synthetic material, a bioabsorbable polymer, a non-woven polymer, other types of polymers, and/or other types of materials or combinations of materials. Examples of suitable biocompatible materials include starch, chitosan, cellulose, agarose, amylose, lignin, hyaluronan, alginate, hyaluronic acid, fibrin glue, fibrin clot, collagen gel, gelatin-resorcin-formalin adhesive, platelet rich plasma (PRP) gel, platelet poor plasma (PPP) gel, Matrigel, Monostearoyl Glycerol co-Succinate (MGSA), Monostearoyl Glycerol co-Succinate/polyethylene glycol (MGSA/PEG) copolymers, laminin, elastin, proteoglycans, polyhydroxybutyrate (PHB), poly(vinyl pyrrolidone) (PVP), polylactide (PLA), polyglycolide (PGA), polycaprolactone (PCL), and their copolymers, non-woven VICRYL® (Ethicon, Inc., Somerville, N.J.), MONOCRYL material, fibrin, non-woven poly-L-lactide, and non-woven PANACRYL (Ethicon, Inc., Somerville, N.J.). Polymers may include aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, poly(propylene fumarate), polyurethane, poly(ester urethane), poly(ether urethane), and blends and copolymers thereof. Suitable synthetic polymers for use in examples described herein may also include biosynthetic polymers based on sequences found in collagen, laminin, glycosaminoglycans, elastin, thrombin, fibronectin, starches, poly(amino acid), gelatin, alginate, pectin, fibrin, oxidized cellulose, chitin, chitosan, tropoelastin, hyaluronic acid, silk, ribonucleic acids, deoxyribonucleic acids, polypeptides, proteins, polysaccharides, polynucleotides, and combinations thereof. Other suitable materials or combinations of materials that may be used will be apparent to those of ordinary skill in the art in view of the teachings herein. It should also be understood that tissue mixed with a scaffold material may have any suitable particle size, and that the resulting mixture may at least initially have the consistency of a slurry or have any other suitable consistency. In some versions, the tissue particles include an effective amount of viable cells that can migrate out of the tissue particle and populate the scaffold. The term “viable,” as used herein, should be understood to include a tissue sample having one or more viable cells.

In some versions, one or more components in a medical fluid or tissue treatment composition comprise one or more healing agents that promote tissue regeneration at a target site (e.g., within a fistula) and/or accelerate tissue healing at the target site. Healing agents may include any of a variety of biocompatible materials that accelerate tissue healing and/or promote tissue regeneration. Such biological components may include, for example, growth factors, proteins, peptides, antibodies, enzymes, platelets, glycoproteins, hormones, cytokines, glycosaminoglycans, nucleic acids, analgesics, viruses, isolated cells, or combinations thereof. The medical fluid may further include one or more additional treatment components that prevent infection, reduce inflammation, prevent or minimize adhesion formation, and/or suppress the immune system. In some versions where a scaffold is used in conjunction with a tissue treatment composition, one or more of these additional biological components or additional treatment components may be provided on and/or within the scaffold. Some examples described herein may also include one or more adhesive agents in conjunction with viable tissue fragments.

Examples described herein relate to the repair (e.g., closing) of lumens in a patient, such as anal fistulas and other types of fistulas. In particular, examples described herein include devices used in at least part of a process to create and/or deliver tissue repair compositions or medical fluid into a lumen such as an anal fistula. It should be understood that anal fistulas and/or other types of fistulas may be relatively difficult to repair (e.g., close) in some settings. The goal of a surgical repair of an anal fistula may be to close the fistula with as little impact as possible on the sphincter muscles. In some settings, a tissue repair composition or medical fluid as described herein may be delivered into the fistula as a liquid composition, a flowable gel or paste, a scaffold plug, or a combination of the two or more of the foregoing (e.g., a porous scaffold plug loaded with a medical fluid composition, etc). Anal fistulas may also be repaired by injecting bioresorbable fibrin glue into the fistula that seals the fistula and promotes tissue growth across the fistula in order to provide permanent closure. Various bioresorbable plugs may also be used to repair anal fistulas. The plug may comprise, for example, collagen protein, tissue, stem cells, and/or other medical fluid components referred to herein; and the plug may be inserted into the fistula where it promotes tissue growth across the fistula as the plug dissolves. If desired, the plug may be secured in place using one or more fasteners and/or one or more adhesive agents. As another merely illustrative example, a medical fluid may be introduced within the fistula, and the medical fluid may eventually harden and then dissolve and/or be absorbed.

Prior to applying a medical fluid to a fistula, it may be desirable in some settings to debride the wall of a fistula (e.g., to remove epithelial cells, etc.), otherwise agitate the wall of the fistula, and/or otherwise treat the walls of the fistula. Merely illustrative examples of how the walls of a fistula may be treated and how a medical fluid may be applied in a fistula will be described in greater detail below. While examples herein are discussed in the context of an anorectal fistula, it should be understood that the following exemplary devices and techniques may be readily applied to various other types of fistulae. Similarly, while the present example relates to treatment of a fistula in a patient, it should also be understood that the following exemplary devices and techniques may be readily applied with respect to various other types of conditions in a patient. Other suitable ways in which the devices and techniques described herein may be used will be apparent to those of ordinary skill in the art in view of the teachings herein.

As used herein, the term “fluid communication” (or in some contexts “communication”) means that there is a path or route through which fluid (gas, liquid or other flowable material) may flow between two components, either directly or through one or more intermediate components. Similarly, the term “conduit” encompasses a conduit within or integrated with a valve. In other words, fluid communication between two components means that fluid can flow from one component to another but does not exclude an intermediate component (e.g., a valve, etc.) between the two recited components that are in fluid communication. Similarly, two or more components may be in mechanical “communication” with each other even if intermediate components are interposed between those two or more components.

II. Exemplary Fistula Repair Device with Fluid Dispensing Barbs

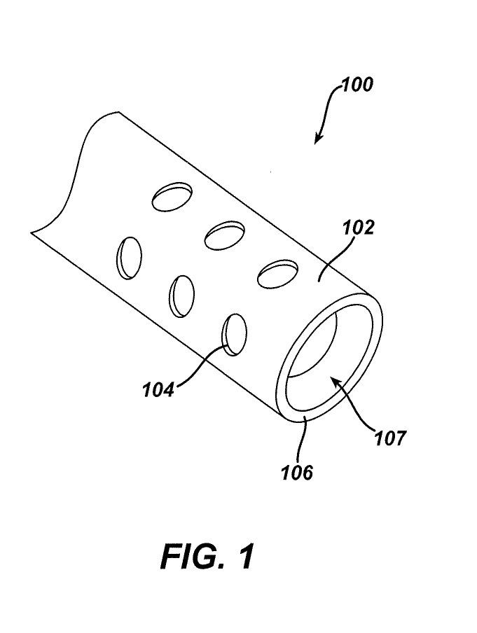

FIGS. 1-4 depict one version of a fistula repair device (100). Fistula repair device (100) of this example comprises an outer sheath (102). Outer sheath (102) has a plurality of openings (104) and a distal end portion (106). Outer sheath (102) is generally cylindrical in shape in the present example, but any other suitable shape may be used as will be apparent to one of ordinary skill in the art in view of the teachings herein. For example, outer sheath (102) may be conically shaped, shaped like a needle, frustoconical in shape, or any other suitable shape. Outer sheath (102) has a hollow construction such that objects may be inserted through outer sheath (102). However, outer sheath (102) may alternatively be at least partially filled rather than hollow in some versions. Outer sheath (102) of the present example has an outer diameter that is small enough to fit inside of a fistula, though sheath (102) may alternatively have any other suitable outer diameter.

Openings (104) are circular in shape in the present example, though any other suitable shape may be used. As shown in FIG. 1, openings (104) are uniformly distributed about the circumference of outer sheath (102). Alternatively, openings (104) may be distributed such that one portion of outer sheath (102) contains a higher concentration of openings (104) whereas another portion of outer sheath (102) has a lower concentration of openings (104), or no openings (104). Each of openings (104) is further shaped to allow at least one of plurality of barbs (108) to extend outward from the inside of outer sheath (102) as will be described in greater detail below. For instance, each opening (104) may communicate with a corresponding recess or trough that is formed through the sidewall of outer sheath (102), with such recesses or troughs further communicating with the hollow interior of outer sheath (102). In some such versions, openings (104) and their corresponding recesses or troughs are each formed by a process of pushing inwardly through the sidewall of outer sheath (102) with a forming tool, providing an inwardly protruding deformation in the interior of the sidewall of sheath (102) at each opening (104) that acts as a catch and redirector for distally advancing barbs (108). In other words, barbs (108) may be guided into and through the recesses or troughs that are associated with openings (104) as barbs (108) are advanced distally, such that barbs (108) ultimately protrude through openings (104).

End portion (106) is generally circular in shape and defines a circular opening (107) in the present example. Opening (107) is substantially perpendicular to the longitudinal axis of outer sheath (102). In some alternative versions, end portion (106) defines an opening having a shape other than a circle. For example, the opening may be slotted, triangular, rectangular, etc. The opening defined by end portion (106) may also be obliquely angled in relation to longitudinal axis of outer sheath (102). As another merely illustrative example, end portion (106) may be closed, such that end portion lacks opening (107) or some variation thereof.

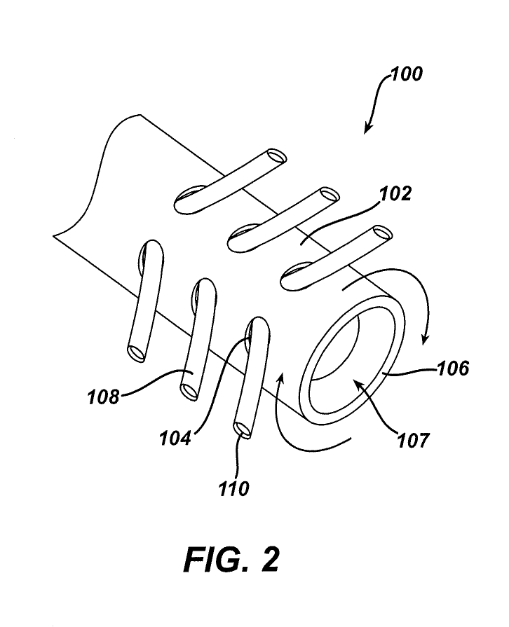

FIG. 2 shows fistula repair device (100) with a plurality of barbs (108) extending outward from outer sheath (102). Barbs (108) have a generally tubular or curved needle shape and may have a cross sectional shape that complements the shape of openings (104). Of course, barbs (108) may have a cross section that is shaped different than openings (104). In some versions, barbs (108) comprise polymer-based micro needles, though it should be understood that barbs (108) may be constructed of any other suitable material or structure. Barbs (108) of the present example have a hollow interior configured to transfer or deliver a mixture of tissue that passes through the interior of each of barbs (108) and exits barbs (108) through a respective barb opening (110). For instance, a medical fluid (112) as described below may be dispensed through barbs (108). In addition, barbs (108) of the present example are bundled within outer sheath (102) when barbs (108) are in a retracted position as shown in FIG. 1. Barbs (108) of the present example are also resiliently biased to extend outwardly as shown in FIG. 2. In particular, as barbs (108) are advanced distally within outer sheath (102), barbs (108) are guided or directed to openings (104) by recesses or troughs in the interior surface of outer sheath (102). Each recess or trough is associated with and leads to a corresponding opening (104), such that once barbs (108) enter the recesses or troughs they will ultimately extend through openings (104) as barbs (108) reach a distal position. In addition or in the alternative to barbs (108) being resiliently biased to extend outwardly (e.g., by shape memory, etc.), the recesses or troughs that are associated with openings (104) may be configured to guide and bend barbs (108) to an outwardly deflected configuration. It should also be understood that barbs (108) may be bundled in several layers of nesting such that two or more sets of barbs (108) may interact with different sets of openings (104) simultaneously.

As depicted in FIG. 2, outer sheath (102) of fistula repair device (100) is configured to rotate. For example, outer sheath (102) may rotate in a clockwise direction or a counter-clockwise direction. Barbs (108) rotate unitarily with outer sheath (102). Barbs (108) are further configured to debride tissue in the walls of a fistula when outer sheath (102) and barbs (108) are rotated after being placed in a fistula tract. In some alternative versions, outer sheath (102) may be configured to move in other ways. For example, outer sheath (102) may be configured to vibrate, reciprocate longitudinally, or engage in other suitable motions. Barbs (108) may be configured to selectively extend and/or retract relative to outer sheath (102). For instance, barbs (108) may be retracted within sheath (102) as sheath (102) is inserted in a fistula; then barbs (108) may be extended relative to sheath (102) to debride tissue and/or dispense a medical fluid (112) in the fistula. The extension or retraction of barbs (108) may occur while outer sheath (102) rotates or may occur serially before or after rotation of outer sheath (102). Furthermore, barbs (108) may be configured to extend and retract in unison, or barbs (108) may be configured to extend and retract independent of each other and/or in selected groups.

In addition to or in lieu of being selectively extended and/or retracted, barbs (108) may be configured to detach from outer sheath (102) after completion of debriding or at any other suitable stage. For instance, fistula repair device (100) may include a barb cutting member (not shown) that may be used to shear or otherwise cut barbs (108) free from outer sheath (102). By way of example only, such a barb cutting member may comprise a tube that has a sharp distal edge and that is translatable relative to outer sheath (102). In some versions, barbs (108) are constructed of a biodegradable material that decomposes inside the tissue forming the wall of a fistula, such that barbs (108) may be safely absorbed by the patient's body regardless of whether detachment of barbs (108) within the patient's tissue is intentional or unintentional. In some such versions, the material forming barbs (108) is also therapeutic. As another merely illustrative variation, barbs (108) may contain medical fluid (112) or some variation thereof when barbs (108) are detached and left in the wall of a fistula, such that barbs (108) continue to deliver medical fluid (112) as barbs (108) are absorbed by the patient's body. Alternatively, the material forming barbs (108) may have any other suitable properties.

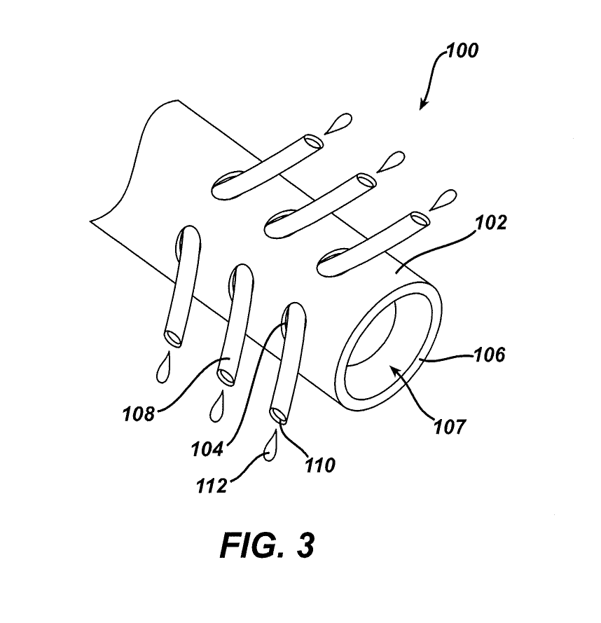

FIG. 3 shows outer sheath (102) with barbs (108) in an extended position and each barb opening (110) releasing medical fluid (112). Medical fluid (112) may be released from all of barbs (108) simultaneously, or barbs (108) may be configured to release medical fluid (112) selectively. Medical fluid (112) may comprise a mixture of biological material such as tissue cells (e.g., stem cells) and an additional biocompatible material. Such an additional biocompatible material may provide a scaffold for tissue in the mixture. Medical fluid (112) may have the consistency of a slurry or any other suitable consistency. Medical fluid (112) may comprise any of the various medical fluid components referred to herein, including combinations of such medical fluid components, and/or any other suitable medical fluid component(s). Medical fluid (112) may be delivered directly in a liquid or semi-liquid form; or medical fluid (112) may be packaged in mini or micro capsules configured to dissolve over time, thus enabling time released delivery of medical fluid (112). Barbs (108) may be configured to deliver medical fluid (112) continually, or alternatively may be configured to deliver therapeutic material in a series of separate deliveries.

In some versions, barbs (108) are repeatedly extended and retracted relative to sheath (102) while in the fistula tract. Such extension and retraction of barbs (108) may form recesses within the tissue wall that defines the fistula tract. These recesses in the tissue wall may then receive medical fluid (112) from openings (110) of barbs (108). For instance, barbs (108) may deliver medical fluid (112) in these recesses while barbs (108) are in an extended position relative to sheath (102) and/or when barbs (108) are in a retracted position relative to sheath (102). In some alternative versions, barbs (108) are formed at least in part of medical fluid (112), and barbs (108) are deployed and left in the fistula and dissolve over time.

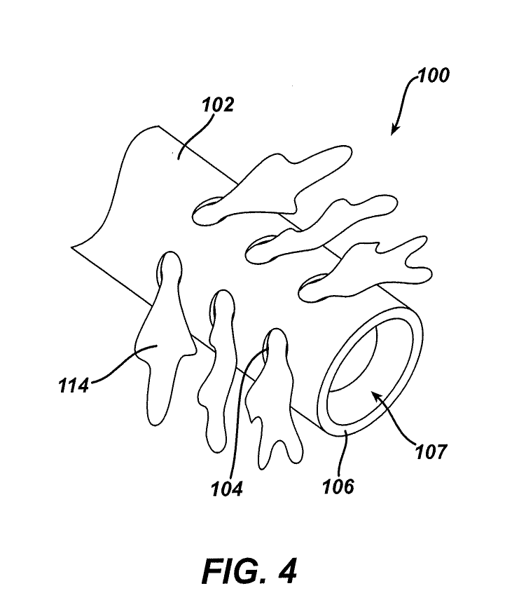

FIG. 4 shows an alternative delivery of a medical fluid (114). Medical fluid (114) may be formulated like any of the formulations of medical fluid (112) described above; or may have any other suitable formulation. Instead of delivering medical fluid (114) through openings (110) of extended barbs (108), medical fluid (114) is released directly through openings (104) of outer sheath (102) in this example. In some versions, releasing medical fluid (114) may be partially carried out with barbs (108) extended and partially carried out without the use of barbs (108). It should also be understood that medical fluid (114) may be delivered through opening (107) of distal end portion (106), in addition to or in lieu of being delivered through openings (104) of outer sheath (102) and/or through openings (110) of extended barbs (108). In some versions where medical fluid (114) is delivered through openings (104), distal end portion (106) is closed, such that sheath (102) lacks opening (107). Other suitable ways in which medical fluid (112, 114) may be delivered will be apparent to those of ordinary skill in the art in view of the teachings herein. It should also be understood that outer sheath (102) may be slowly withdrawn from the fistula tract as medical fluid (112, 114) is being delivered, such that outer sheath (102) “covers its tracks” with medical fluid (112, 114) as outer sheath (102) is being withdrawn from the fistula.

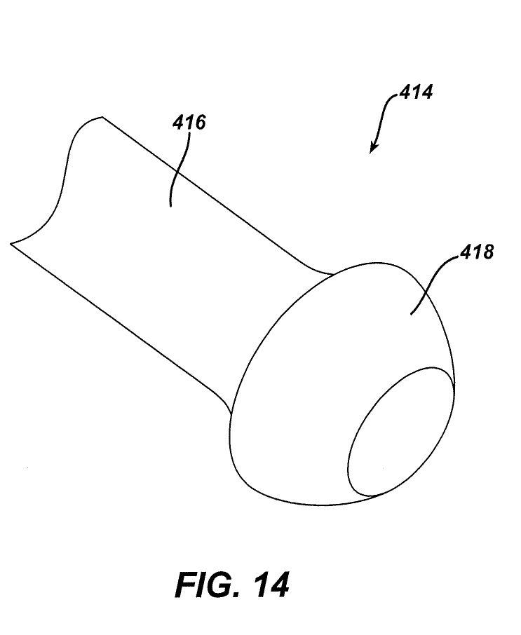

In some versions, regardless of how medical fluid (114) is delivered to the target site (e.g., a fistula tract), medical fluid (114) begins coagulating after it is delivered. As medical fluid (114) coagulates, medical fluid (114) may form a plug (414), as shown in FIG. 14, in the fistula where medical fluid (114) is applied. Plug (414) may comprise a stem portion (416) and a cap portion (418). Stem portion (416) may be generally cylindrical in shape, and may generally conform to the shape of the fistula as a result of medical fluid (114) coagulating within the fistula. Stem portion (416) further forms in the fistula in a way that helps prevent inadvertent slipping of plug (414) from the fistula and is configured to aid in the repair of the fistula due to the therapeutic composition of plug (414). Furthermore, as medical fluid (114) coagulates within the fistula, a portion of medical fluid (114) that is exposed relative to the fistula (e.g., a portion of medical fluid (114) that seeps out the distal end of the fistula) may coagulate to form cap portion (418). Cap portion (418) in this example has a knob-like or mushroom shaped structure, which has a larger diameter than stem portion (416). Cap portion (418) also has a diameter larger than the width of the fistula where plug (414) is formed. Cap portion (418) may be positioned so as to abut the entrance of the fistula to prevent plug (414) from undesirably sliding or shifting through the fistula; and to provide a better seal to prevent leakage of body fluids, etc.

In some versions, cap portion (418) may be formed by the release of medical fluid (114) in conjunction with use of a forming device (not shown). Forming device may comprise an anvil or other structure capable of shaping medical fluid (114). The user may determine which end of the fistula tract that the user desires to form cap portion (418) of plug (414). Then, the user may hold the forming device against or near the opening at that end of the fistula tract to catch medical fluid (114) as medical fluid (114) is released into the fistula. The forming device may have a knob-like or hemispherical shaped recess such that when medical fluid (114) coagulates, medical fluid (114) coagulates in a knob-like or mushroom shape to form cap portion (418). Once medical fluid (114) is fully, or nearly fully coagulated, the forming device may be removed, thus leaving cap portion (418) properly formed. Other suitable ways of forming plug (414) will be apparent to one of ordinary skill in the art in view of the teachings herein.

III. Exemplary Fistula Repair Device with Integral Stent

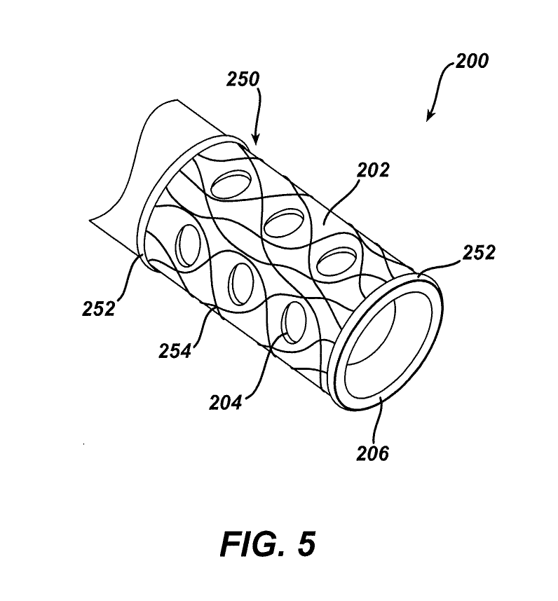

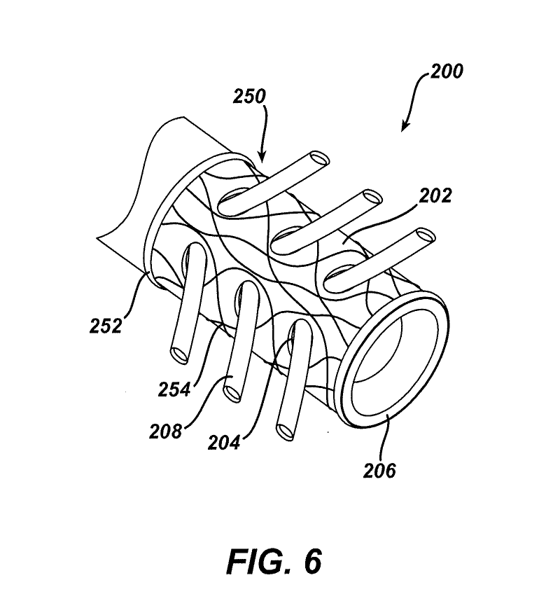

FIGS. 5-6 depict an exemplary alternative version of a fistula repair device (200), which comprises a stent (250), an outer sheath (202), a plurality of openings (204), and a distal end portion (206). Stent (250) is constructed so as to circumferentially fit around and be removably attached to outer sheath (202). Furthermore, stent (250) is configured to be placed inside the body, and more particularly inside a fistula as will be described in greater detail below. Stent (250) comprises a mesh (254) and end rings (252) in the present example. It should be understood that end rings (252) are merely optional. Stent (250) has a generally cylindrical shape formed by mesh (254), and end rings (252) define the ends of the generally cylindrical shape of stent (250) in the present example. Of course, any other suitable shape for stent (250) may be used. Mesh (254) wraps around outer sheath (202) between end rings (252) of stent (250). Mesh (254) is configured to have structural integrity even when not wrapped around outer sheath (202), such that stent (250) may be inserted into a fistula and left in the fistula, without sheath (202) providing structural support and without stent (250) collapsing. Mesh (254) is configured and positioned such that mesh (254) does not substantially interfere with openings (204) when stent (250) is mounted to sheath (202). Thus, a medical fluid may be released through openings (204) without substantial physical interference from stent (250). Stent (250) may be constructed of a material configured to safely decompose or degrade over time when placed inside the body.

FIG. 6 shows fistula repair device (200) with stent (250) having barbs (208) in an extended position. Stent (250) is positioned such that barbs (208) may extend through openings (204) outward from outer sheath (202) without substantially interfering with mesh (254). Barbs (208) may further retract into outer sheath (202) without interfering with mesh (254) of stent (250). Mesh (254) may comprise an abrasive coating or texture such that mesh (254) may facilitate debriding of tissue from the wall of a fistula. For instance, fistula repair device (200) may be inserted into a fistula and then be rotated and/or longitudinally reciprocated within the fistula in order to debride the fistula wall with mesh (254). Alternatively, mesh (254) may comprise a smooth coating. Barbs (208) may be configured and operable in a manner similar to the various configurations and operabilities of barbs (108) described above. Thus, barbs (208) may be used to debride the wall of a fistula, create recesses for the receipt of a medical fluid in the wall of a fistula, and/or deliver a medical fluid to the fistula. In addition or in the alternative, barbs (208) may be integrally formed with stent (250), such that stent (250) may be held in place at least in part by barbs (208) extending outward from stent (250) into walls of a fistula, thus substantially securing stent (250) within the fistula.

IV. Exemplary Fistula Repair Device for Use with Endoscope

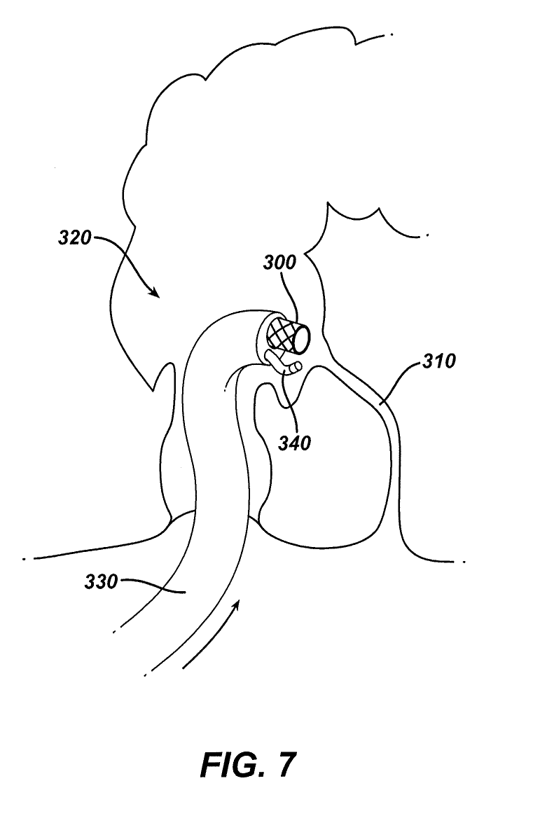

FIGS. 7-12 depict an exemplary method of using a fistula repair device (300) for therapeutic cell delivery to a fistula (310). FIG. 7 depicts an endoscope (330) transanally inserted in the rectum (320) of a patient. Fistula repair device (300) is slidably inserted through a working channel of endoscope (330) and protrudes from the distal end of endoscope (330). Endoscope (330) includes visualization optics (340) that are configured to enable a user to view fistula (310) to assist in guiding fistula repair device (300) into fistula (310). Endoscope (330) is flexible to enable endoscope (330) to bend toward fistula (310) after insertion of endoscope (330) into rectum (320). Accordingly, at least part of fistula repair device (300) and at least part of visualization optics (340) may also be constructed to be flexible to flex with endoscope (330). In some versions, endoscope (330), fistula repair device (300), and visualization optics (340) may be configured to be selectively flexible by the user such that the user can control the flexing of endoscope (330), fistula repair device (300), and visualization optics (340) remotely.

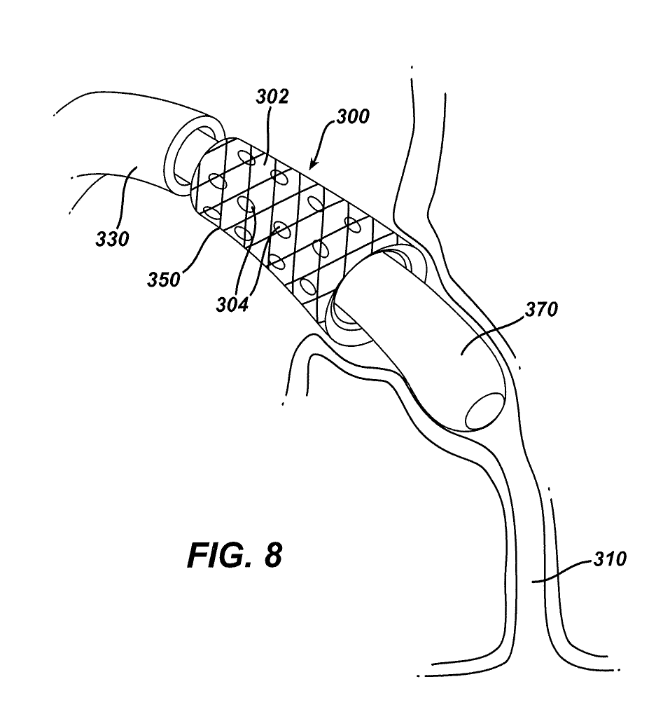

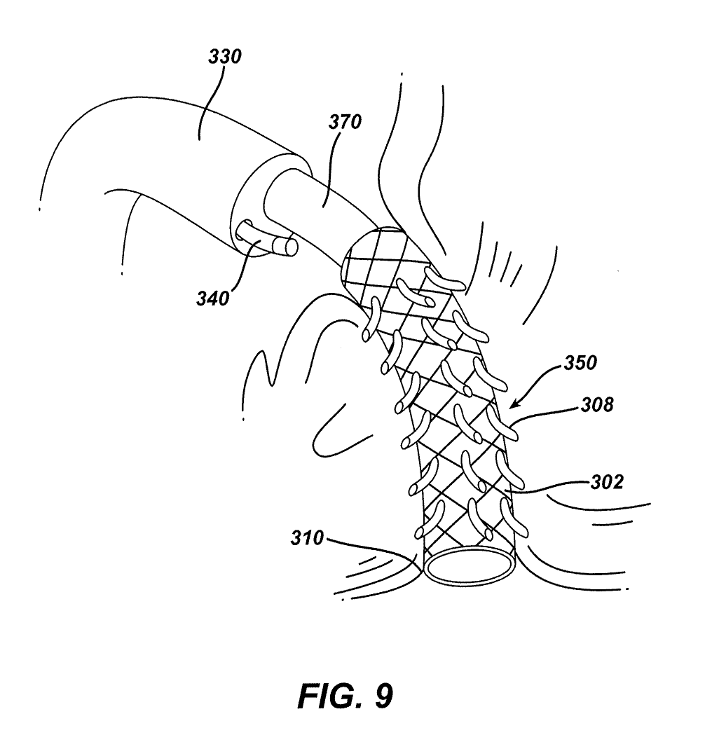

FIG. 8 depicts endoscope (330) near an entrance of fistula (310). In the illustrated version, fistula repair device (300) comprises a stent (350) surrounding an outer sheath (302) having a plurality of openings (304). Outer sheath (302) is positioned about a catheter (370), which extends from the end of endoscope (330). It should be understood that fistula repair device (300) may be configured and operable in a manner substantially similar to the configuration and operability of fistula repair device (200) described above. As shown in FIG. 8, catheter (370) is first inserted into fistula (310) to guide outer sheath (302) and stent (350) into fistula (310). In the process of extending into fistula (310), catheter (370) stretches the wall of fistula (310) so as to provide room for stent (350). The distal end of catheter (370) may be rounded and/or tapered to facilitate such insertion and stretching. As catheter (370) extends further into fistula (310), sheath (302) and stent (350) may begin insertion into fistula (310). FIG. 9 depicts sheath (302) and stent (350) inserted into fistula (310). In this example, sheath (302) and stent (350) have a length that is approximately equal to the length of fistula (310), such that sheath (302) and stent (350) extend the full length of fistula (310). Alternatively, sheath (302) and/or stent (350) may have any other suitable length. Similarly, while catheter (370) extends along the full length of sheath (302) and stent (350) at this stage, catheter (370) may instead extend to any other suitable length. Visualization optics (340) may be used to determine if stent (350) has been properly positioned in fistula (310) at this stage.

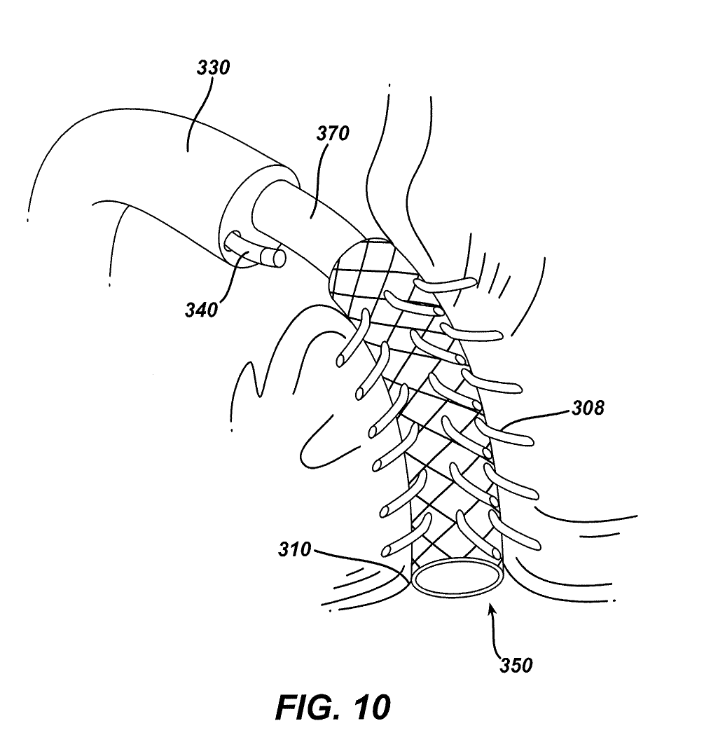

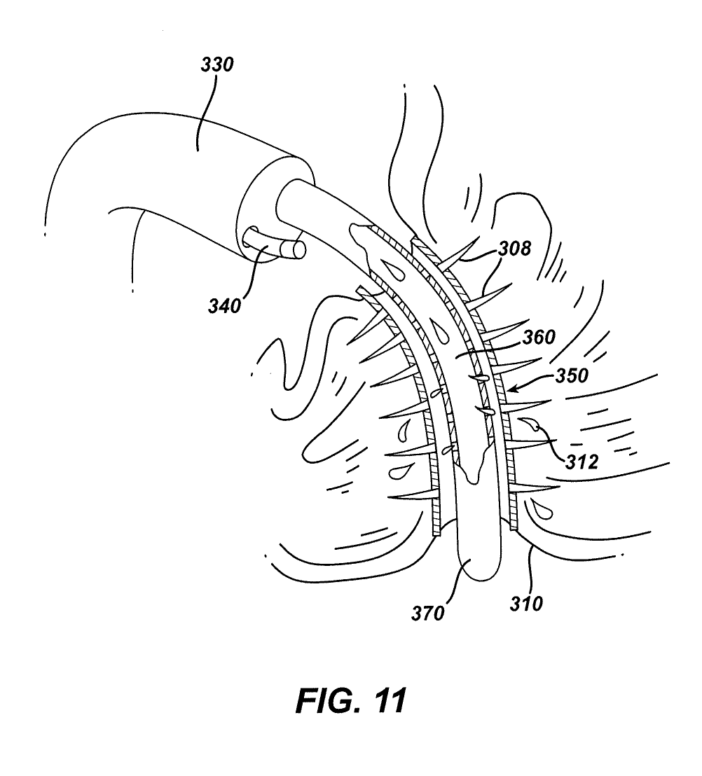

With sheath (302) and stent (350) being sufficiently inserted in fistula (310), barbs (308) are partially extended into the wall of fistula (310) as shown in FIG. 9. Barbs (308) may then be used to debride the wall of fistula (310). For instance, catheter (370) may be rotated and/or reciprocated longitudinally to rotate and/or reciprocate barbs (308) within fistula (310). In some versions, stent (350) may be used to debride the wall of fistula (310), in addition to or in lieu of using barbs (308) to debride the wall of fistula (310). It should also be understood that a fluid medium such as saline, etc., may be communicated to fistula (310) at this stage to assist in flushing away of epithelial cells that have been removed as part of the debriding process. In the present example, once the wall of fistula (310) is sufficiently debrided, barbs (308) are extended further into walls of fistula (310) as shown in FIGS. 10-11. Such extension of barbs (308) creates recesses in the tissue of the wall of fistula (310) to accept medical fluid (312).

In some versions, barbs (308) are formed by a middle tube that comprises a plurality of relatively small diameter tubes coupled together around a main middle tube radius, such that the small diameter tubes collectively form the middle tube. Such a middle tube may be disposed within sheath (302). Each small diameter tube may have a pair of associated longitudinal slits and an associated transverse slit. These slits may allow a free end formed by each group of slits to separate outwardly from the middle tube that is formed by the small diameter tubes. Such free ends may form barbs (308), and may be positioned at various locations along the length of the middle tube. When the middle tube is in a retracted position within sheath (302), the free ends may be constrained within sheath (302). However, when the middle tube is advanced relative to sheath (302), the free ends pass through openings (304), forming outwardly extending barbs (308). In addition, proximal portions of barbs (308) may further separate from the middle tube as the middle tube is advanced distally to flare barbs (308) outwardly. Various other suitable ways in which barbs (308) may be formed and/or actuated will be apparent to those of ordinary skill in the art in view of the teachings herein.

As best seen in FIG. 11, catheter (370) is further configured to deliver a medical fluid (312) to stent (350) such that an amount of medical fluid (312) exits barbs (308) into the walls of fistula (310). Medical fluid (312) may be formulated like any of the formulations of medical fluid (112) described above; or may have any other suitable formulation. In some versions, catheter (370) delivers medical fluid (312) through barbs (308). For instance, barbs (308) may be hollow with open free ends that are configured to dispense medical fluid (312), similar to barbs (108) described above. In addition or in the alternative, medical fluid (312) may be communicated from openings formed in catheter (370) through openings (304) of outer sheath (302). As yet another merely illustrative variation, medical fluid (312) may be communicated directly through a port of endoscope (330) or through a separate dispensing tube that is inserted through endoscope (330). Still other suitable ways in which medical fluid (312) may be delivered to fistula (310) will be apparent to those of ordinary skill in the art in view of the teachings herein. It should also be understood that barbs (308) and/or the middle tube that forms barbs (308) may be made of a bioabsorbable material (e.g., a bioabsorbable plastic) that may be left in the fistula (310) with stent (350).

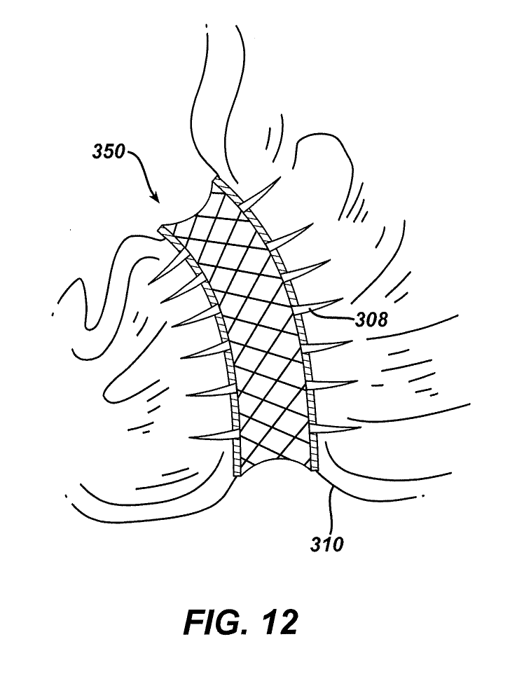

As is also shown in FIG. 12, catheter (370) has been retracted from stent (350) at this stage. In addition, sheath (302) is retracted into endoscope (330). In some versions, catheter (370) is selectively expandable or inflatable to selectively secure sheath (302) and/or stent (350) to catheter (370). For instance, catheter (370) may be in an expanded configuration before and during insertion of sheath (302) and stent (350) in fistula (310), to substantially secure the longitudinal position of sheath (302) and stent (350) along catheter (370) during insertion in fistula (310). After sheath (302) and stent (350) have been sufficiently inserted into fistula (310), catheter may be at least partially collapsed or deflated to leave sheath (302) and stent (350) in place within fistula (310). Various other suitable relationships between catheter (370), sheath (302), and stent (350) will be apparent to those of ordinary skill in the art in view of the teachings herein.

As shown in FIG. 12, endoscope (330) and visualization optics (340) have also been removed from near the entrance of fistula (310). Stent (350) has been properly positioned in fistula (310), and in the illustrated version, fistula (310) is held open by stent (350). In some other alternative versions, fistula (310) may be sealed, rather than held open by stent (350), as will be discussed further below. In the present example, barbs (308) are also left in fistula (310). As noted above, barbs (308) may assist in securing stent (350) in place. As also noted above, barbs (308) may be formulated of a therapeutic bioabsorbable material in some versions as well. Alternatively, barbs (308) may have any other suitable properties. It should also be understood that barbs (308) may be retracted relative to stent (350) and be withdrawn along with endoscope (330) and catheter (370), etc., when stent (350) is left in place in fistula (310). In some such versions, stent (350) may remain in fistula (310) along with remaining medical fluid (312) that was dispensed by fistula repair device (300). In addition to or in lieu of leaving stent (350) in fistula (310), a suture or other structure may be applied by fistula repair device (300) to provide a “seton” or “seton stitch.” Such a seton may thus be combined with medical fluid (312). The material forming a seton stitch may even be impregnated with a medical fluid (312) if desired. It should also be understood that stent (350) may be formed of a bioabsorbable material, and that one or more medical fluids may be provided within the interior cavity of stent (350) and/or as a coating on/in stent (350). To the extent that stent (350) maintains passageway through the fistula for at least a temporary period, it should be understood that maintaining such a passageway may facilitate the drainage of bodily fluids from the fistula, etc. Various other suitable ways in which fistula repair device (300) may be made and used will be apparent to those of ordinary skill in the art in view of the teachings herein.

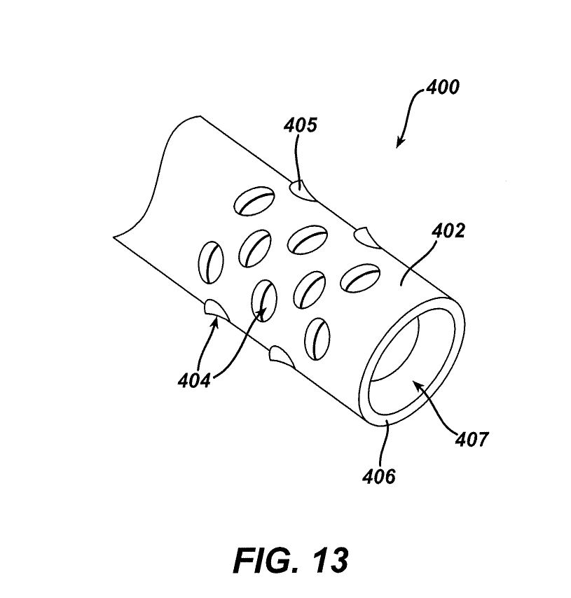

FIG. 13 depicts another exemplary version of a fistula repair device (400). Fistula repair device (400) of this example comprises an outer sheath (402), which has a plurality of abrasive openings (404) and a distal end portion (406). Outer sheath (402) is generally cylindrical in shape in the present example, though it should be understood that any other suitable shape may be used. For example, outer sheath (402) may be conically shaped, shaped like a needle, frustoconical in shape, or any other suitable shape. Outer sheath (402) has a hollow construction such that objects may be inserted through outer sheath (402). However, outer sheath (402) may alternatively be at least partially filled rather than hollow in some versions. As with other examples described herein, outer sheath (402) of the present example has an outer diameter that is small enough to fit inside of a fistula, though sheath (402) may alternatively have any other suitable outer diameter.

Abrasive openings (404) are circular in shape and are positioned substantially evenly across the surface of outer sheath (402) in the present example. Alternatively, abrasive openings (404) may be located on selected portions of surface of outer sheath (402). Each abrasive opening (404) has an associated protruding portion (405) that is configured to scrape or debride tissue by rubbing against the tissue. In particular, each protruding portion (405) has a sharp edge operable to agitate or cut off small portions of tissue, such as epithelial cells. For example, such debriding may be accomplished by rotating outer sheath (402) about its longitudinal axis and/or reciprocating outer sheath (402) along its longitudinal axis when outer sheath (402) is inserted adjacent to tissue (e.g., within a fistula tract, etc.). Agitated tissue may then be removed or mixed and reapplied. In some versions, a textured surface is provided adjacent to abrasive openings (404). End portion (406) is generally circular in shape and defines a circular opening (407) in the present example. Opening (407) is substantially perpendicular to the longitudinal axis of outer sheath (402). In some alternative versions, end portion (406) defines an opening having a shape other than a circle. For example, the opening may be slotted, triangular, rectangular, etc. The opening defined by end portion (406) may also be obliquely angled in relation to longitudinal axis of outer sheath (402).

Fistula repair device (400) may be used in a manner similar to any other fistula repair device described herein. For instance, fistula repair device (400) may be inserted into a fistula, either from within the patient's rectum, through an external opening of the fistula, or otherwise. Fistula repair device (400) may then be rotated about its longitudinal axis and/or reciprocated along its longitudinal axis to debride epithelial cells from the wall of fistula with protruding portions (405). In some settings, such debriding of epithelial cells may allow healing to occur to eventually close the fistula. Next, a medical fluid such as any medical fluid described herein may be dispensed in the fistula through openings (404) and/or opening (407). If barbs are present in fistula repair device (400), such barbs may be extended and/or retracted as part of the process of debriding and/or as part of the process of dispensing medical fluid. Fistula repair device (400) may then be withdrawn from the fistula. The medical fluid may congeal to form a plug within the fistula, similar to plug (414) shown in FIG. 14. Other suitable ways in which fistula repair device (400) may be used will be apparent to those of ordinary skill in the art in view of the teachings herein.

V. Exemplary Fistula Repair Device with Integral Balloon

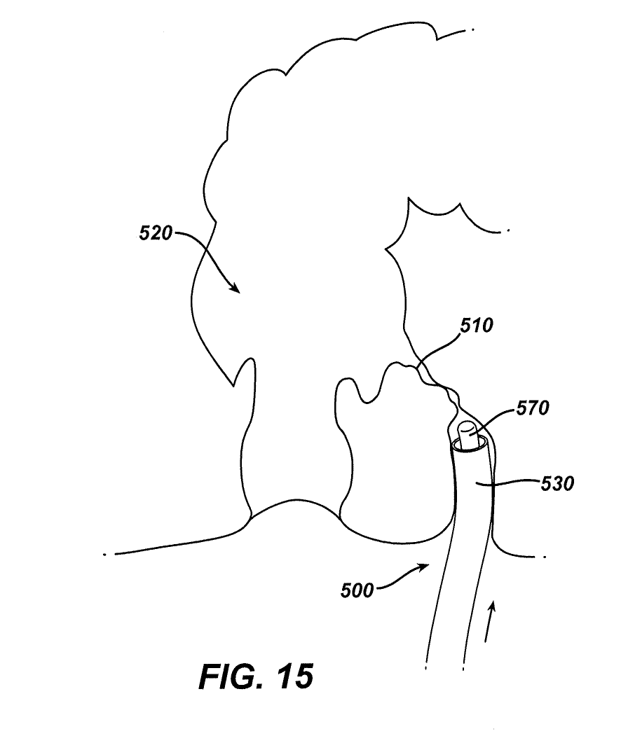

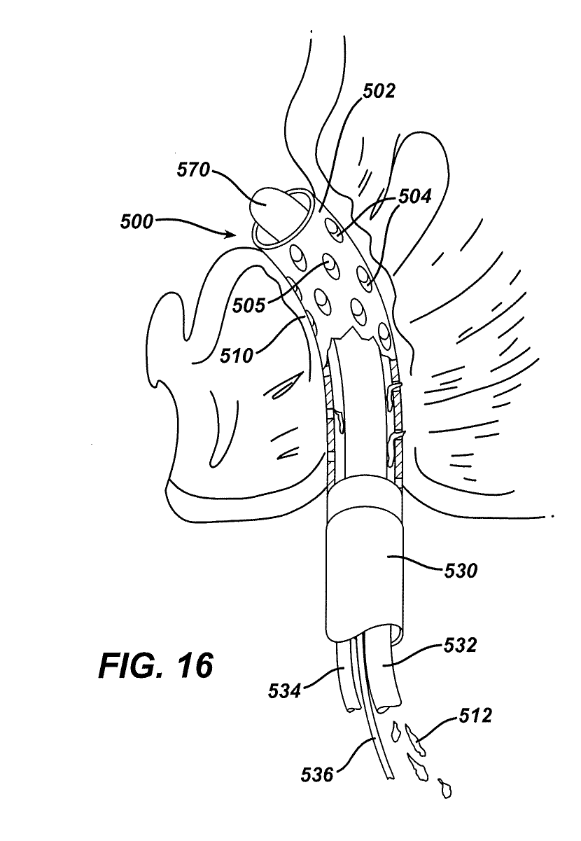

FIGS. 15-18 depict another exemplary method of using a fistula repair device (500). Fistula repair device (500) comprises an outer tube (530) with a catheter (570) inserted through outer tube (530). As can be seen in FIG. 16, fistula repair device (500) of this example further comprises an outer sheath (502), which includes a plurality of openings (504). Catheter (570) provides a flexible shaft and is configured to guide outer sheath (502) through fistula (510). Similar to outer sheath (402) of fistula repair device (400) described above, outer sheath (502) of fistula repair device (500) also includes protruding portions (505) adjacent to each opening (504). Protruding portions (505) are configured to scrape or debride tissue by rubbing against the tissue. In particular, each protruding portion (505) has a sharp edge operable to agitate or cut off small portions of tissue, such as epithelial cells. For example, such debriding may be accomplished by rotating outer sheath (502) about its longitudinal axis and/or reciprocating outer sheath (502) along its longitudinal axis. Protruding portions (505) may also harvest tissue from fistula (510) for use with a medical fluid (514) as described below. Tissue harvested from fistula (510) may reside within the interior of outer sheath (502) until it is mixed with liquid in fistula (510) to form medical fluid/slurry (514). Alternatively, tissue harvested from fistula (510) may be communicated proximally through outer tube (530) and/or catheter (570), be mixed with liquid to form medical fluid (514) at some location other than within fistula (510), then be communicated distally with medical fluid (514) back into fistula (510). Other suitable ways in which harvested tissue and/or medical fluid (514) may be handled will be apparent to those of ordinary skill in the art in view of the teachings herein.

FIG. 15 depicts fistula repair device (500) being inserted through an external opening of the fistula (510) rather than being inserted into the fistula via the rectum (520). Of course, fistula repair device (500) may instead be inserted into the fistula (510) via the rectum (520). FIG. 16 depicts a closer view of fistula repair device (500) inserted into fistula (510). In the illustrated version, outer tube (530) is positioned near the entrance of fistula (510) while catheter (570) is used to guide outer sheath (502) through fistula (510). Outer sheath (502) is inserted into fistula (510) such that protruding portions (505) are positioned to debride the wall of fistula (510) upon rotation and/or translation of outer sheath (502) within fistula (510). A vacuum tube (532) may be inserted inside outer sheath (502) and may be configured to suck out loosened epithelial cells (512) and/or other debris that is loosened during the debriding process. In addition or in the alternative, the epithelial cells (512) and other debris that is loosened during this debriding process may be flushed with saline or some other liquid, which may be communicated through openings (504) of outer sheath (502) or otherwise.

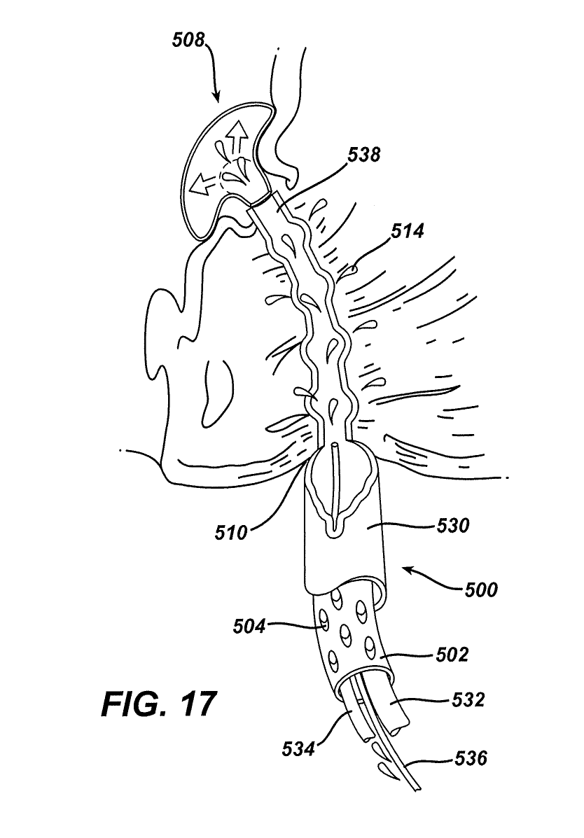

A dispensing tube (534) is inserted inside outer sheath (502) to deliver medical fluid (514) to fistula (510) through abrasive holes (504); or alternatively, as shown in FIG. 17, medical fluid (514) may be delivered directly to fistula (510) after outer sheath (502) is retracted. Medical fluid (514) may be formulated like any of the formulations of medical fluid (112) described above; or may have any other suitable formulation. A balloon filling tube (536) is also positioned inside outer sheath (502). As shown in FIG. 17, a balloon (508) is located at the distal end of fistula repair device (500). In some versions, balloon (508) is formed by the distal end of catheter (570). In some other versions, balloon (508) is carried by the distal end of catheter (570). In the present example, balloon (508) is inflated with liquid such as saline or any other suitable fluid via balloon filling tube (536). Once inflated, balloon (508) of the present example defines an umbrella-like shape as can be seen in FIG. 17, though balloon (508) may alternatively define any other suitable shape when inflated.

While FIG. 17 shows outer sheath (502) in a retracted position relative to fistula (510) while balloon (508) is being inflated, it should be understand that outer sheath (502) may instead be at least partially disposed in fistula (510) while balloon (508) is being inflated. In addition, during or after the process of inflating balloon (508), medical fluid (514) is delivered within fistula (510) via dispensing tube (534). Inflated balloon (508) may substantially prevent such medical fluid (514) from leaking out of an end of fistula (510). In some versions, medical fluid (514) already contains biological material such as minced tissue when it is communicated through dispensing tube (534). In some other versions, fluid that is communicated through dispensing tube (534) consists solely of a fluid medium (e.g., fibrin, PRP, etc.) that will mix with minced tissue already residing in outer sheath (502). Such minced tissue already residing in outer sheath (502) may have been harvested by outer sheath (502) as described above.

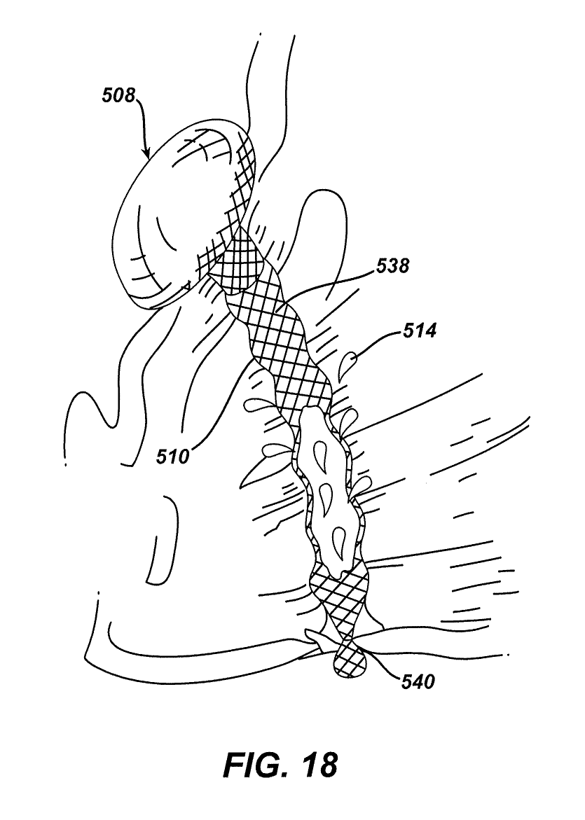

FIG. 18 shows fistula (510) filled with medical fluid (514), with outer sheath (502) and other components of fistula repair device (500) having been withdrawn from fistula (510), and with inflated balloon (508) remaining at one end of fistula (510). Inflated balloon (508) thus provides a cap at one end of fistula (510) while sealed tissue (540) provides a seal at the other end of fistula (510). Sealed tissue (540) may be sealed using any of the devices or techniques described herein for sealing an end of a fistula; or using any other suitable device or technique. Medical fluid (514) within fistula (510) at this stage eventually sets or congeals, forming a therapeutic plug (538). Balloon (508) may then be deflated and removed. Alternatively, balloon (508) may be left in place until it eventually sloughs off or degrades, etc.

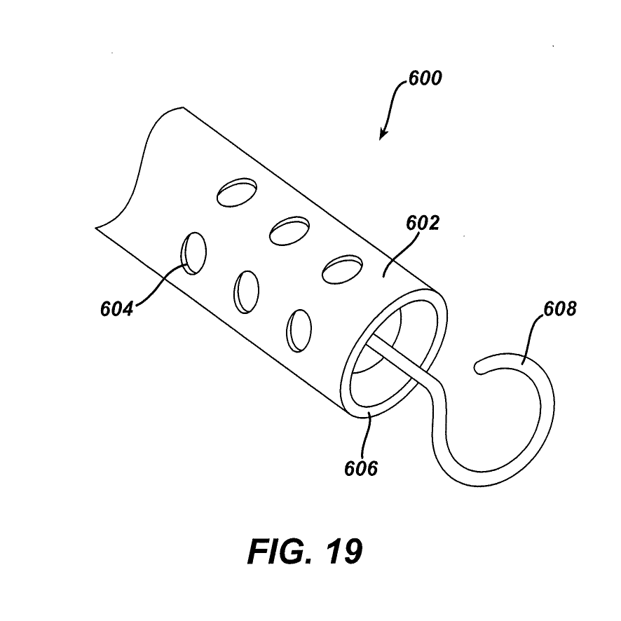

FIG. 19 depicts another exemplary fistula repair device (600). Fistula repair device (600) comprises an outer sheath (602) having a plurality of openings (604) and an open distal end portion (606). Except as noted below, fistula repair device (600) of this example has a substantially identical structure and operability as fistula repair device (100) described above. However, fistula repair device (600) of this example further comprises a mechanical tissue closure hook (608). Closure hook (608) extends through the center of outer sheath (602) and protrudes distally through open distal end portion (606). While closure hook (608) of the present example has a hook-like shape, it should be understood that any other suitable shape may be used. Closure hook (608) is configured to be actuated by a user, such as by selectively extending or retracting closure hook (608) relative to outer sheath (602) and/or by selectively rotating closure hook (608) relative to outer sheath (602). When inserted in a fistula, closure hook (608) is operable to tie together tissue at the end of the fistula. In some versions, at least a portion of closure hook (608) is left in place like a suture. In some such versions, closure hook (608) is formed at least in part of a biodegradable material, such that a portion of closure hook (608) that is left in place may degrade and essentially disappear over time. In some versions, closure hook (608) is merely used to hold tissue together in an apposed configuration while the user applies an adhesive or other means of binding and/or sealing tissue. It should be understood that closure hook (608) may be used prior to injection of a medical fluid in a fistula. In other words, closure hook (608) may be used to keep an end of the fistula closed as medical fluid is injected in the fistula, thereby preventing leakage of the medical fluid from the fistula before the medical fluid has a chance to coagulate, gel, and/or be absorbed, etc. Other suitable ways in which closure hook (608) may be configured and used will be apparent to those of ordinary skill in the art in view of the teachings herein.

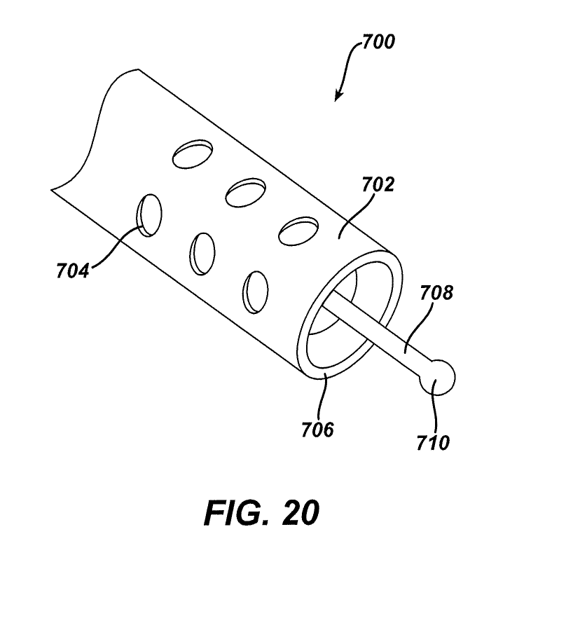

FIG. 20 depicts yet another exemplary fistula repair device (700). Fistula repair device (700) comprises an outer sheath (702) having a plurality of openings (704) and an open distal end portion (706). Except as noted below, fistula repair device (700) of this example has a substantially identical structure and operability as fistula repair device (100) described above. However, fistula repair device (700) of this example further comprises a radio frequency (RF) sealer (708). RF sealer (708) extends through the center of outer sheath (702) and protrudes distally through open distal end portion (706). RF sealer (708) is configured to be actuated by a user, such as by selectively extending or retracting closure hook (608) relative to outer sheath (602) and/or by selectively activating RF sealer (708) with RF energy. In the present example, RF sealer (708) is monopolar, though RF sealer (708) may be bipolar if desired. RF sealer (708) has a rod-like shape with a contact knob (710) at the distal end of RF sealer (708), though any other suitable configurations may be used. Contact knob (710) is configured to transmit radio frequency energy operable to heat nearby tissue thus binding the tissue and providing a seal when applied to, for example, tissue at the end of a fistula. Other suitable ways in which RF sealer (708) may be configured and used will be apparent to those of ordinary skill in the art in view of the teachings herein.

VI. Exemplary Fistula Repair Device with Sealing Member

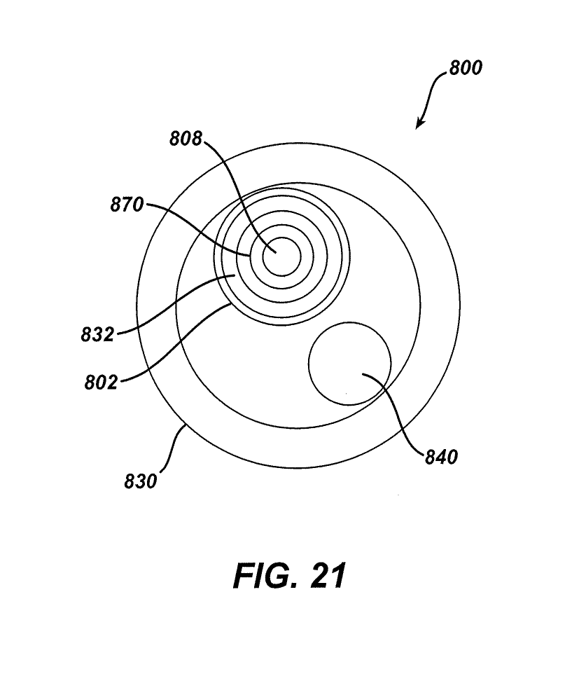

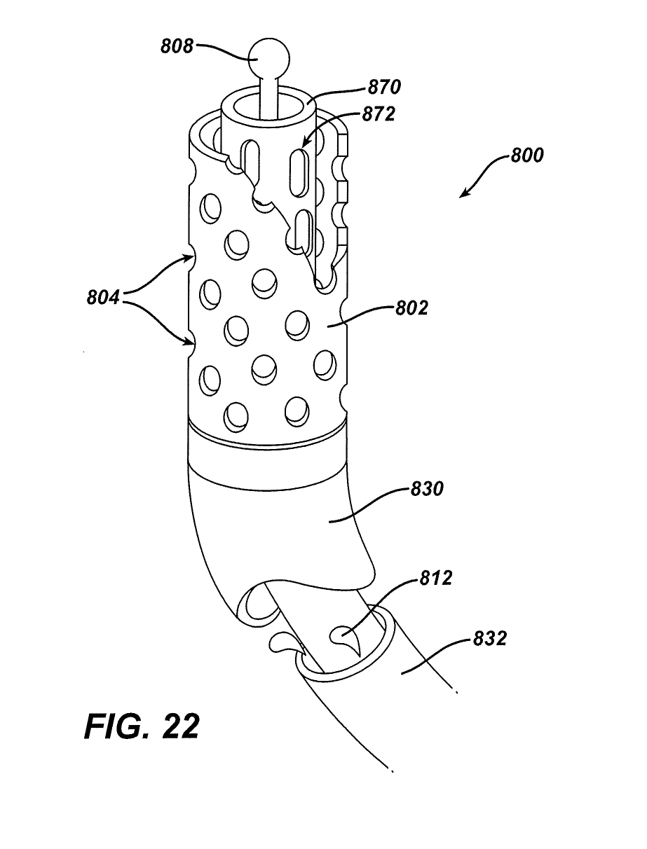

FIGS. 21-26 show another exemplary fistula repair device (800). As best seen in FIGS. 21-22, fistula repair device (800) of this example comprises an outer sheath (802) and a delivery tube (832) located within outer sheath (802). Outer sheath (802) is operable to debride a fistula tract, and delivery tube (832) is configured to deliver medical fluid to the fistula, as will be described in greater detail below. A vacuum catheter (870) is located inside of delivery tube (832), and an RF sealer (808) is located inside of vacuum catheter (870). RF sealer (808), vacuum catheter (870), delivery tube (832), and outer sheath (802) are all coaxial with each other and are all positioned within a working channel of an endoscope (830) in the present example. It should be understood, however, that each of these components may be repositioned or reconfigured in any suitable manner. For example, vacuum catheter (870) may be positioned on the inner most tube of the nested tubes. In addition, fistula repair device (800) may be used without an endoscope (830) in some versions.

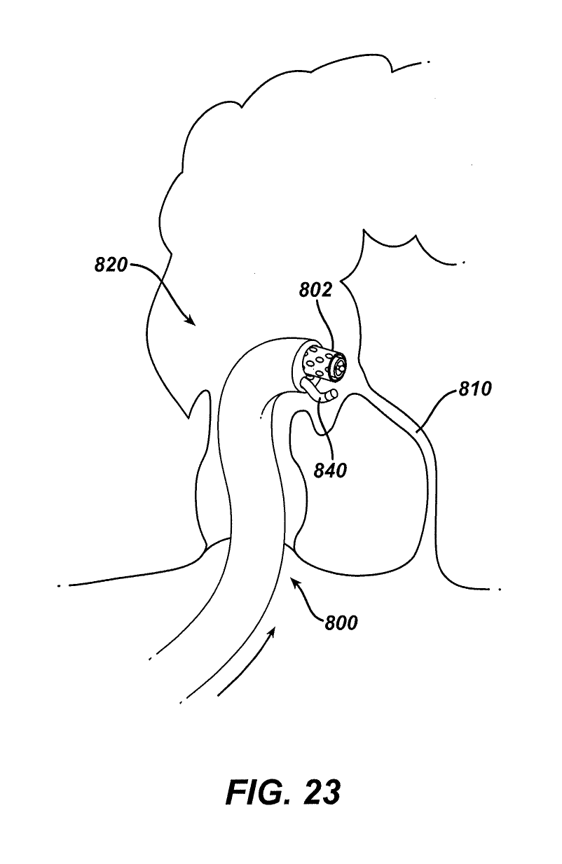

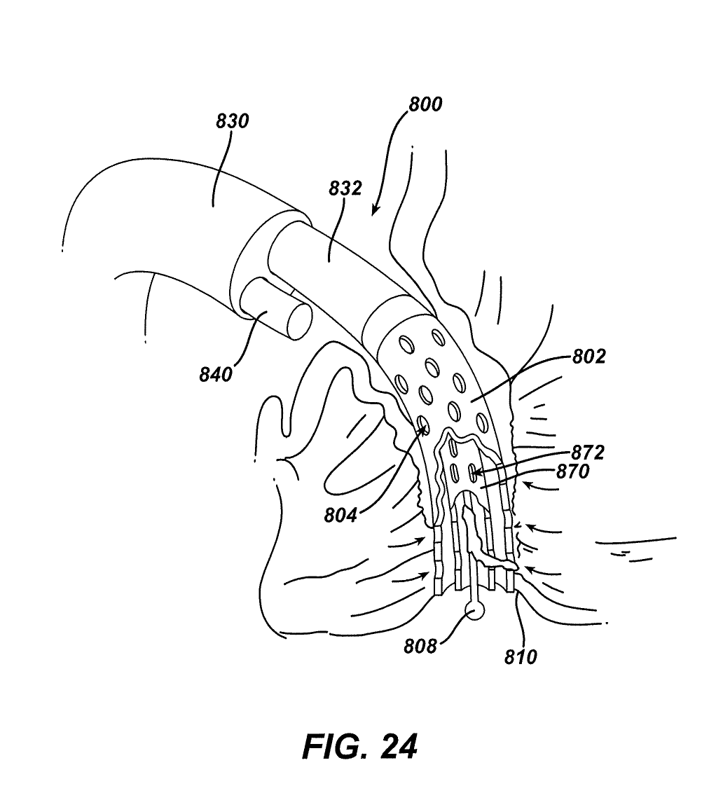

FIGS. 23-26 depict an exemplary method of using fistula repair device (800) to repair a fistula (810). As shown in FIG. 23, endoscope (830) is inserted through the rectum (820) of a patient to reach an interior end of fistula (810). Endoscope (830) houses fistula repair device (800) and visualization optics (840) that are configured to enable a user to view the route of fistula repair device (800) from rectum (820) to fistula (810), enabling the user to position endoscope (830) and fistula repair device (800) properly. With endoscope (830) and fistula repair device (800) suitably positioned, fistula repair device (800) is inserted into fistula (810) as shown in FIG. 24. As outer sheath (802) is inserted into fistula (810), openings (804) on outer sheath (802) are used to debride the wall of fistula (810) as described elsewhere herein with similar openings in similar outer sheaths. Vacuum catheter (870) may then be used to remove loose debrided tissue from fistula (810). In particular, a vacuum communicated through vacuum catheter (870) may be further communicated through openings (872) formed in vacuum catheter (870); with the vacuum being further communicated through openings (804) of outer sheath (802). In some versions, a liquid such as saline is used to flush loose debrided tissue, in addition to or in lieu of using a vacuum. In some versions, tissue harvested from fistula (810) using fistula repair device (800) is mixed with a medical fluid (812) and then reapplied to fistula (810).

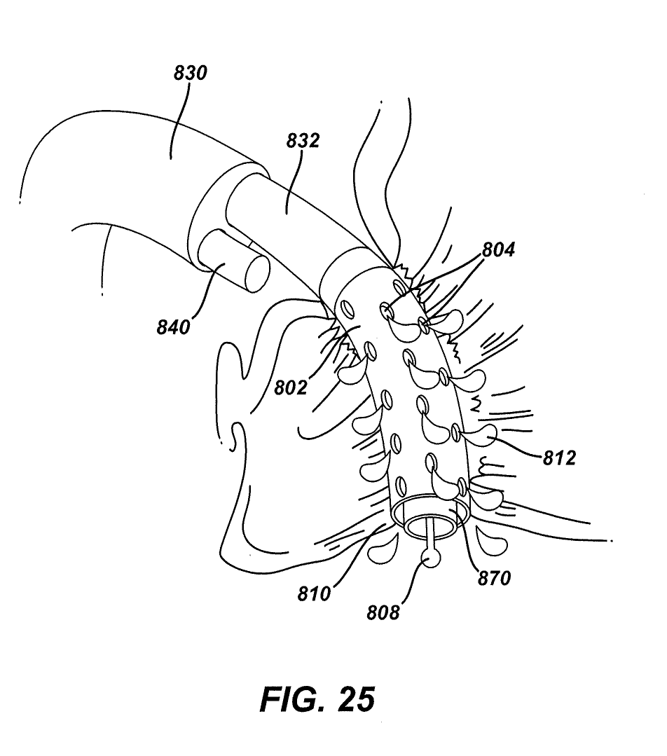

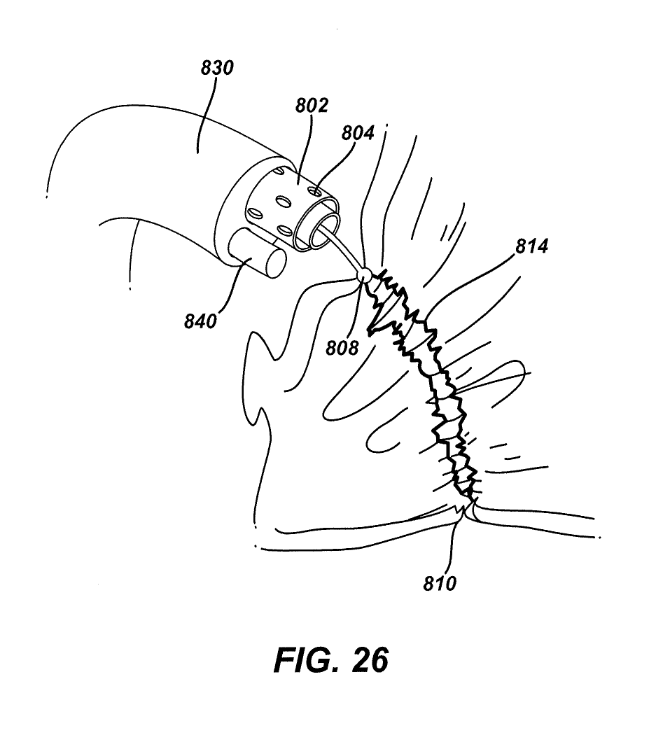

Once fistula (810) has been sufficiently debrided, and once a sufficient amount of tissue has been harvested from fistula (810) (to the extent that tissue is even harvested from fistula (810)), delivery tube (832) is used to apply medical fluid (812) within fistula (810) as shown in FIG. 25. Medical fluid (812) may be formulated like any of the formulations of medical fluid (112) described above; or may have any other suitable formulation. Medical fluid (812) exits outer sheath (802) through holes (804). After a sufficient amount of medical fluid (812) is applied to fistula (810), RF sealer (808) may be activated to seal each end fistula (810), as shown in FIG. 26. RF sealer (808) may be configured and operable in a manner similar to the configurations and operabilities of RF sealer (708) described above; or RF sealer (808) may have any other suitable configuration and operability. For instance, in the present example, RF sealer (808) seals fistula (810) closed as RF sealer (808) is retracted through fistula (810). In some versions, RF sealer (808) is used to seal the exterior opening of fistula (810) before fistula repair device (800) is inserted in the interior opening of fistula (810). Then RF sealer (808) is used to seal the interior opening of fistula (810) after a sufficient amount of medical fluid (812) has been dispensed in fistula (810). Alternatively, the user may wait until a sufficient amount of medical fluid (812) has been dispensed in fistula (810) before sealing the exterior opening of fistula (810). Other suitable ways in which fistula repair device (800) may be configured and used will be apparent to those of ordinary skill in the art in view of the teachings herein.

VII. Miscellaneous

While several devices and components thereof have been discussed in detail above, it should be understood that the components, features, configurations, and methods of using the devices discussed are not limited to the contexts provided above. In particular, components, features, configurations, and methods of use described in the context of one of the devices may be incorporated into any of the other devices. Furthermore, not limited to the further description provided below, additional and alternative suitable components, features, configurations, and methods of using the devices, as well as various ways in which the teachings herein may be combined and interchanged, will be apparent to those of ordinary skill in the art in view of the teachings herein.

Versions of the devices described above may be actuated mechanically or electromechanically (e.g., using one or more electrical motors, solenoids, etc.). However, other actuation modes may be suitable as well including but not limited to pneumatic and/or hydraulic actuation, etc. Various suitable ways in which such alternative forms of actuation may be provided in a device as described above will be apparent to those of ordinary skill in the art in view of the teachings herein.

Versions of the devices described above may have various types of construction. By way of example only, any of the devices described herein, or components thereof, may be constructed from suitable metals, ceramics, plastics, or combinations thereof. Furthermore, although not required, the construction of devices described herein may be configured to be compatible with or optimize their use with various imaging technologies. For instance, a device configured for use with MRI may be constructed from all non-ferromagnetic materials. Also for instance, when using optional imaging technologies with devices described herein, certain configurations may include modifications to materials of construction such that portions or the device may readily appear in a resultant image. Various suitable ways in which these and other modifications to the construction of devices described herein may be carried out will be apparent to those of ordinary skill in the art in view of the teachings herein.

Versions of the devices described above may have application in conventional medical treatments and procedures conducted by a medical professional, as well as application in robotic-assisted medical treatments and procedures.

Versions of described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various versions in the present disclosure, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometries, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus comprising:
(a) a tissue agitation member configured to fit inside of a fistula, wherein the tissue agitation member is further configured to agitate tissue located on a wall of a fistula upon movement of the tissue agitation member within the fistula; and
(b) a delivery member, wherein the delivery device is in communication with the tissue agitation member, wherein the delivery member is configured to communicate a medical fluid from within the tissue agitation member to a wall of a fistula.

2. The apparatus of claim 1, wherein the tissue agitation member comprises a plurality of barbs.

3. The apparatus of claim 2, wherein the tissue agitation member further comprises a sheath, wherein the barbs are selectively extendable or retractable relative to the sheath.

4. The apparatus of claim 2, wherein at least one of the barbs comprises a hollow tubular needle structure configured to communicate the medical fluid.

5. The apparatus of claim 1, further comprising a stent in communication with tissue agitation member, wherein the stent is configured to be inserted into a fistula.

6. The apparatus of claim 1, further comprising a balloon, wherein the balloon is configured to block an end of a fistula.

7. The apparatus of claim 1, wherein the tissue agitation member comprises a plurality of openings defined by abrasive edges.

8. The apparatus of claim 1, wherein the tissue agitation member is configured to debride tissue located on a wall of a fistula upon movement of the tissue agitation member within the fistula.

9. The apparatus of claim 1, further comprising a sealer, wherein the sealer is configured to seal an end of a fistula.

10. The apparatus of claim 9, wherein the sealer comprises a radio frequency sealer configured to seal an end of a fistula by using radio frequency energy.

11. A method of repairing a fistula using a fistula repair device, wherein the fistula repair device comprises a tissue debriding member and a delivery member, wherein the delivery member is in communication with at least one source of medical fluid, the method comprising:
(a) inserting at least a portion of the fistula repair device into the fistula;
(b) debriding the walls of the fistula with the tissue debriding member;
(c) administering the medical fluid through the delivery member to the fistula; and
(d) removing the fistula repair device from the fistula.

12. The method of claim 11, wherein the tissue debriding member comprises a plurality of barbs, wherein the act of debriding the walls of the fistula further comprises:
(i) extending the barbs from the fistula repair device, and
(ii) rotating or reciprocating the tissue debriding member within the fistula.

13. The method of claim 11, further comprising removing epithelial cells from the fistula loosened by the act of debriding, wherein the act of epithelial cells comprises one or both of evacuating the epithelial cells with a vacuum or flushing the epithelial cells with a liquid.

14. The method of claim 11, further comprising inserting a catheter is into at least a portion of the fistula to stretch out at least a portion of the fistula before inserting the fistula repair device into the fistula.

15. The method of claim 11, wherein the medical fluid comprises a mixture of harvested tissue and a scaffold material.

16. A method of repairing a fistula using a fistula repair device, wherein the fistula repair device comprises a tissue debriding member, a delivery member configured to deliver a medical fluid to the fistula, and a stent, the method comprising:
(a) inserting at least a portion of the fistula repair device into the fistula;
(b) positioning the stent in the fistula;
(c) debriding the walls of the fistula with the tissue debriding member;
(d) delivering the medical fluid to the fistula;
(e) removing the tissue debriding member from the fistula; and
(f) leaving the stent positioned in the fistula.

17. The method of claim 16, wherein the fistula repair device further comprises a plurality of selectively extendable barbs, the method further comprising extending the barbs into a wall of the fistula.

18. The method of claim 17, wherein the barbs are hollow, wherein the act of delivering the medical fluid to the fistula comprises communicating the medical fluid through the barbs.

19. The method of claim 16, wherein the stent is formed of a bioabsorbable material.

20. The method of claim 16, wherein the stent further comprises mesh walls, wherein the mesh walls are configured to hold the fistula open.
